(19) United States
(12) Patent Application Publication
(10) Pub. No.: US 2019/0247435 A1
(43) Pub. Date: Aug. 15, 2019

(54) NEOANTIGENS AS TARGETS FOR IMMUNOTHERAPY

(71) Applicant: The Johns Hopkins University, Baltimore, MD (US)

(72) Inventors: Victor Velculescu, Dayton, MD (US); Valsamo Anagnostou, Baltimore, MD (US)

(21) Appl. No.: 16/312,152

(22) PCT Filed: Jun. 23, 2017
(86) PCT No.: PCT/US2017/038942

§ 371 Date: Dec. 20, 2018

Related U.S. Application Data
(60) Provisional application No. 62/356,107, filed on Jun. 29, 2016.

(51) Int. Cl. A61K 35/17; C12Q 1/6886; C12Q 1/6869; C12Q 1/6881; C07K 14/74; A61K 39/39; A61P 35/00

(52) U.S. Cl. CPC A61K 35/17; C12Q 1/6886; C12Q 1/6869; A61P 35/00; C07K 14/70539; A61K 39/39; C12Q 1/6881

(57) ABSTRACT

Immune checkpoint inhibitors have shown significant therapeutic responses against tumors containing increased mutation-associated neoantigen load. We have observed the emergence of acquired resistance in non-small cell lung cancer patients that were initially responsive to immune checkpoint blockade. Resistance occurred 4-11 months after the initiation of immunotherapy and both clinical response and therapeutic resistance were associated with changes in T cell clonality but not with changes in expression of PD-L1. Genomic analyses of responsive and resistant tumors from the same patients identified loss of 7 to 18 mutation-associated putative neoantigens in resistant clones that were predicted to have high MHC binding affinity. Neoantigen loss occurred through elimination of tumor subclones or through deletion of chromosomal regions containing truncal alterations. These analyses provide insights into the mechanisms of evasion to immune checkpoint blockade and immune therapies that target tumor neoantigens.

This invention was made with government support under grant number CA121113 awarded by National Institutes of Health. The government has certain rights in the invention.

### TECHNICAL FIELD OF THE INVENTION

This invention is related to the area of cancer. In particular, it relates to immunotherapy for cancer.

### BACKGROUND OF THE INVENTION

Tumor cells contain non-synonymous somatic mutations that alter the amino acid sequences of the proteins encoded by the affected genes¹. Those alterations are foreign to the immune system and may therefore represent tumor-specific neoantigens capable of inducing anti-tumor immune responses². Somatic mutational and neoantigen density has recently been shown to confer long-term benefit from immune checkpoint blockade in non-small cell lung cancer (NSCLC)³ and melanoma^{4,5} suggesting that neoepitopes stemming from somatic mutations may he critical for deriving clinical benefit from immunotherapy. Expression of the programmed cell death ligand 1 (PD-L1) in tumors or tumor-infiltrating immune cells have been associated with responses to PD-1 blockade⁶⁻⁸, however PD-L1 expression or other immune biomarkers have not been sufficient to fully explain therapeutic outcomes.

Among the patients that initially respond to PD-1 blockade, some become resistant to the therapy. Up-regulation of alternate immune checkpoints⁹, loss of HLA haplotypes¹⁰ or somatic mutations in HLA genes¹¹ have been proposed as mechanisms of evasion to immune recognition in some patients, but the mechanisms underlying response and acquired resistance to immune checkpoint blockade have remained elusive.

There is a continuing need in the art to identify more effective ways for treating cancers and avoiding relapse.

### SUMMARY OF THE INVENTION

One aspect of the invention is a method of identifying target epitopes for a tumor of an individual. Massively parallel sequencing is performed on a first sample of the individual comprising tumor DNA, on a second sample from the individual comprising normal tissue DNA, and on a third sample from the individual comprising tumor DNA. The first sample is obtained prior to treatment with an anti-tumor agent and the third sample is obtained after treatment with the anti-tumor agent. Somatic mutations in the first sample that encode a different amino acid sequence than in the second sample and form mutant epitopes are identified. The mutant epitopes in the first sample are analyzed to identify epitopes that are recognized by class I MHC molecules of a type expressed by the individual. From among the epitopes that are recognized by class I MHC molecules of the type expressed by the individual, a first particular mutant epitope that is absent in the third sample is identified, and from among the same epitopes a second particular mutant epitope that is present in the third sample is identified.

An aspect of the invention is a personalized, anti-tumor immunogenic preparation for an individual cancer patient who initially responded to anti-tumor therapy and later became resistant to the therapy. The preparation comprises a peptide that comprises a mutant epitope, and an adjuvant. The mutant epitope is expressed in a tumor in the individual cancer patient. The mutant epitope is recognized by a class I MHC molecule expressed by the individual cancer patient. The mutant epitope is present in the tumor after the tumor became resistant to the therapy.

Another aspect of the invention is a personalized, anti-tumor, chimeric antigen receptor (CAR) for an individual cancer patient who initially responded to anti-tumor therapy and later became resistant to the therapy. The CAR comprises a single chain variable region fragment that specifically binds to a mutant epitope. The mutant epitope is expressed in a tumor in the individual cancer patient. The mutant epitope is recognized by a class I MHC molecule expressed by the individual cancer patient. The mutant epitope is present in the tumor after the tumor became resistant to the therapy.

Yet another aspect of the invention is a personalized, anti-tumor, chimeric antigen receptor T cell for an individual cancer patient who initially responded to anti-tumor therapy and later became resistant to the therapy. The personalized, anti-tumor, chimeric antigen receptor T cell comprises a chimeric antigen receptor (CAR). The CAR comprises a single chain variable region fragment that specifically binds to a mutant epitope. The mutant epitope is expressed in a tumor in the individual cancer patient. The mutant epitope is recognized by a class I MHC molecule expressed by the individual cancer patient. The mutant epitope is present in the tumor after the tumor became resistant to the therapy.

Still another aspect of the invention is a method of identifying target epitopes for a tumor of an individual. Massively parallel sequencing is performed on a first liquid biopsy sample of the individual comprising tumor DNA and on a second liquid biopsy sample from the individual comprising tumor DNA; the first sample is obtained prior to treatment with an anti-tumor agent and the second sample is obtained after treatment with the anti-tumor agent. Somatic mutations are identified in the first sample that encode a different amino acid sequence than encoded by normal DNA of the individual and that form mutant epitopes. The mutant epitopes in the first sample are analyzed to identify epitopes that are recognized by class I MHC molecules of a type expressed by the individual. From among the epitopes that are recognized by class I MHC molecules of the type expressed by the individual a first particular mutant epitope is identified that is absent in the second sample and a second particular mutant epitope is identified that is present in the second sample.

These and other embodiments which will be apparent to those of skill in the art upon reading the specification provide the art with methods for identifying useful personalized targets for cancer patients and associated reagents for treating the cancer patients.

### BRIEF DESCRIPTION OF THE DRAWINGS

FIG. 1. Overview of next-generation sequencing and neoantigen prediction analyses. Whole exome sequencing was performed on the pre-treatment and post-progression tumor and matched normal samples. Exome data were applied in a neoantigen prediction pipeline that evaluates antigen processing, MHC binding and gene expression to generate neoantigens specific to the patient's HLA haplotype. Truncal neoantigens were identified by correcting for tumor purity and ploidy and the TCR repertoire was evaluated at baseline, at the time of response and upon emergence of resistance.

FIGS. 2A-2I. Emergence of resistance to immune checkpoint blockade is associated with elimination of mutation associated neoantigens by loss of heterozygosity and a more diverse T-cell repertoire independent of PD-L1 expression. FIG. 2A shows computed tomographic (CT) images of patient CGLU117 at baseline, at the time of therapeutic response and at time of acquired resistance. Pre-treatment CT image of the abdomen, demonstrates a right adrenal mass (T1, circled), radiologic tumor regression is noted after 2 months of treatment, followed by disease relapse at 4 months from treatment initiation with a markedly increased right adrenal metastasis (T2, circled). 3^{rd} follow up CT demonstrates further disease progression in the adrenal lesion. Tumor burden kinetics for target lesions by RECIST criteria are shown in FIG. 2B. Peripheral T cell expansion of a subset of intratumoral clones was noted to peak at the time of response and decrease to baseline levels at the time of resistance (FIG. 2C). Productive TCR frequency denotes the frequency of a specific rearrangement that can produce a functional protein receptor among all productive rearrangements. FIG. 2D and FIG. 2E show B allele frequency graphs for chromosome 17, a value of 0.5 indicates a heterozygous genotype whereas allelic imbalance is observed as a deviation from 0.5. The region that undergoes loss of heterozygousity (LOH) in the resistant tumor (FIG. 2E, box) contains 3 mutation associated neoantigens that are thus eliminated. No differences in CD8-+ T cell density (FIG. 2F, FIG. 2G) or PD-L1 expression (FIG. 2H, FIG. 2I) were observed between baseline and resistant tumors.

FIG. 3 (Table 1.) Characteristics of Eliminated Neoantigens

### DETAILED DESCRIPTION OF THE INVENTION

Loss of certain neoantigens is important for the acquisition of resistance to anti-tumor agents including checkpoint blockade agents such as anti-PD-1 and anti-PD-L1 antibodies. These certain neoantigens are present in cancer cells of individuals prior to treatment. However, the mutations creating these certain neoantigens are present among a population of more numerous mutations. If mutations creating these certain neoantigens can be identified, specific targeting agents can be made for one or more of the certain neoantigens. These specific targeting agents can be used therapeutically alone or in conjunction with the anti-tumor agents, such as checkpoint blockade agents.

The certain neoantigens and relevant neoepitopes can be identified using one or more methods. In one method, somatic mutations are identified. These can typically be found by comparing tumor to non-tumor DNA. These can be found in known tumor and known normal tissues. Alternatively, a liquid biopsy, e.g., from plasma or stool, may contain both tumor and normal DNA so that a separate normal sample need not be obtained and analyzed. For neoantigen identification, coding sequences can be selectively screened. When mutations are found, non-synonymous mutations should be selected. Cellularity of mutations may be determined, with mutations that are found in a high percentage of the cells forming a desirable category. When greater than 75% of the cells have a particular mutation it may be considered truncal. Neoepitopes for a particular MHC haplotype can be determined, in particular for a MHC haplotype of the individual. Binding affinity of the neoepitopes and the MHC molecules can be analyzed. Processing, self-similarity, and gene expression of the neoantigens or neoepitopes can be analyzed.

Any type of massively parallel sequencing may be used. These include without limitation pyrosequencing, sequencing by reversible terminator chemistry, sequencing by ligation mediated by ligase enzymes, and phospholinked fluorescent nucleotides or real time sequencing. Templates for sequencing may be prepared by any available technique including without limitation, emulsion PCR, clonal bridge amplification, and gridded DNA-nanoballs. In some techniques, a single molecule of template is sequenced. Any of the techniques as are known in the art may be used.

Any technique known in the art for determining binding to MHC class I molecules may be used. One such method is a pan-allele/pan-length algorithm Neilsen et al., Genome Med. 2016, 8:33. In another method a peptide sequence is threaded onto a template, based on a crystal structure. Interaction energy is calculated for each position of a peptide and they are summed for the whole peptide. Schueler-Furman et al., Protein Science 2000, 9:1838-46.

The MHC class I type of a patient may be determined according to standard means. Each person carries two alleles of each of the three class-I genes, (HLA-A, HLA-B and HLA-C). A person can express six different types of MHC-I. HLA testing can be performed on a sample of blood from the patient, particularly on lymphocytes. HLA typing can be determined, for example, by testing the HLA proteins on the surface of white blood cells or by testing DNA from the same cells.

Expression level of a neoantigen may be performed using any known analysis of protein or mRNA, for example. Various quantitative methods can be performed, as is convenient. Methods for measuring expression levels of RNA include northern blotting, RT-qPCR, quantitative PCR on an array, hybridization microarray, serial analysis of gene expression, RNA-Seq. Methods for measuring expression levels of protein include Western blot, enzyme-linked immunosorbent assay. Any method as is conveient can be used.

Any type of mutation which forms a neoepitope may be of interest. These include those that are non-synonymous, including single amino acid substitutions, frame shift mutations, and small insertions or deletions of from 1-5 amino acid residues.

Once a neoepitope is identified, and optionally verified as one that has good MHC affinity, and high cellularity, it can be used as the target of various specific immunotherapies. A peptide vaccine can be made for immunizing the patient to stimulate an immune response to the tumor. Peptides comprising neoepitopes may be at least 6, 10, 15, 20, 25, 30, 25, 40, 50, 60, 70, 80, or 90 amino acid residues and may be less than 500, 400, 300, 200, or 100 amino acid residues. Peptides can be made by any method known in the art, including without limitation, synthetic chemistry, solid phase peptide synthesis, recombinant organism synthesis, and isolation and purification from natural sources. The peptide vaccine may be administered with other substances, including immune adjuvants, checkpoint inhibitors, etc. Any immune adjuvant known in the art may be used. Exemplary adjuvants include aluminum salts, squalene, MF59 and QS21.

When loss of a neoepitope occurs upon acquisition f resistance, such a neoepitope can be used as a vaccine element to prevent reoccurrence. It can be used in combination with a neoepitope that is not lost upon acquired resistance. Alternatively, a retained neoepitope can be used alone. Alternatively treatment with these types of neoepitopes can be alternating and/or cycled.

The peptide comprising the neoantigen epitope may be linked, e.g., covalently, non-covalently, or as a fusion protein, with another protein, peptide, or chemical agent. Other peptides to which it may be linked may be those that are known to enhance an immune response. Peptides may be synthesized, for example, on a solid support, in recombinant organisms, or using an automatic synthesis program.

Other types of therapies that can be prepared and administered targeting the neoepitope that is identified include adoptive T cell transfer and CAR T cell transfer. In adoptive T cell transfer T cells of the patient are withdrawn and stimulated in vitro with the target peptide. They can be expanded in vitro prior to infusing back to the same patient. Thus the patient's own T cells are stimulated specifically for the neoantigen or neoepitope outside of the body and used therapeutically to target the neoantigen or neoepitope inside the body. CAR T cells can be made by constructing a chimeric antigen receptor using a single chain variable fragment and one or more co-stimulation domains. As in adoptive T cell transfer, lymphocytes may be obtained from the patient and they can be modified in vitro. In this case they are modified by introduction of a nucleic acid from which the chimeric receptor can be expressed. The single chain variable fragment may be derived from a monoclonal antibody that specifically binds to the neoantigen or neoepitope. The variable portions of a monoclonal antibody's immunoglobulin heavy and light chain may be fused together via a linker to form a scFv. This scFv may be preceded by a signal peptide for proper localization. A transmembrane domain may he used to connect the extracellular scFv portion to the intracellular co-stimulation domain(s). Co-stimulation domains may be obtained from CD32-zeta, CD28, and/or OX40.

Anti-tumor agents include without limitation chemotherapy agents and immunotherapy agents. The latter category include checkpoint blockade agents. These may be anti-CTLA-4, anti-PD-L1, ipilimumab, tremelimumab, anti-PD-1, anti-PD-L2, nivolumab, pembrolizumab, anti-LAG3, anti-B7-H3, anti-B7-H4, anti-TIM3. Typically these agents are antibodies or antibody derivatives. Loss of neoantigens associated with treatment with other anti-tumor antibodies may also be identified and used. Chemotherapeutic agents which may be used include without limitation Abitrexate (Methotrexate injection); Abraxane (Paclitaxel Injection); Adcetris (Brentuximab Vedotin Injection); Adriamycin (Doxorubicin); Adrucil Injection (5-FU (fluorouracil)); Afinitor (Everolimus); Afinitor Disperz (Everolimus); Alimta (PEMETREXED); Alkeran Injection (Melphalan Injection); Alkeran Tablets (Melphalan); Aredi a (Pamidronate); Arimidex (Anastrozole); Aromasin (Exemestane); /library/breast; Arranon (Nelarabine); Arzerra (Ofatumumab Injection); Avastin (Bevacizumab); Beleodaq (Belinostat Injection); Bexxar (Tositumomab); BiCNU (Carmustine); Blenoxane (Bleomycin); Blincyto (Blinatumomab Injection); Bosulif (Bosutinib); Busulfex Injection (Busulfan Injection); Campath (Alemtuzumab); Camptosar (Irinotecan); Caprelsa (Vandetanib); Casodex (Bicalutamide); CeeNU (Lomustine); CeeNU Dose Pack (Lomustine); Cerubidine (Daunorubicin); Clolar (Clofarabine Injection); Cometriq (Cabozantinib); Cosmegen (Dactinomycin); Cotellic (Cobimetinib); Cyramza (Ramucirumab Injection); CytosarU (Cytarabine); Cytoxan (Cytoxan); Cytoxan Injection (Cyclophospharnide Injection); Dacogen (Decitabine); DaunoXome (Daunorubicin Lipid Complex Injection); Decadron (Dexamethasone); DepoCyt (Cytarabine Lipid Complex Injection); Dexamethasone Intensol (Dexamethasone); Dexpak Taperpak (Dexamethasone); Docefrez (Docetaxel); Doxil (Doxorubicin Lipid Complex Injection); Droxia (Hydroxyurea); DTIC (Decarbazine); Eligard (Leuprolide); Ellence (Ellence (epirubicin)); Eloxatin (Eloxatin (oxaliplatin)); Elspar (Asparaginase); Emcyt (Estramustine); Erbitu.x (Cetuximab); Erivedge (Vismodegib); Erwinaze (Asparaginase Erwinia chrysanthemi); Ethyol (Amifostine); Etopophos (Etoposide Injection); Eulexin (Flutamide); Fareston (Toremifene); Farydak (Panobinostat); Faslodex (Fulvestrant); Femara (Letrozole); Firmagon (Degarelix Injection); Fludara (Fludarabine); Folex (Methotrexate Injection); Folotyn (Pralatrexate Injection); FUDR (FUDR (floxuridine)); Gazyva (Obinutuzumab Injection); Gemzar (Gemcitabine); Gilotrif (Afatinib); Gleevec (Imatinib Mesylate); Gliadel Wafer (Carmustine wafer); Halaven (Eribulin injection); Herceptin (Trastuzumab); Hexalen (Altretamine); Hycamtin (Topotecan); Hycamtin (Topotecan); Hydrea (Hydroxyurea); Ibrance (Palbociclib); Iclusig (Ponatinib); Idamycin PFS (Idarubicin); Ifex (Ifosfamide); Imbruvica (Ibrutinib); Inlyta (Axitinib); Intron A alfab (Interferon alfa-2a); Iressa (Gefitinib); Istodax (Romidepsin Injection); Ixempra (Ixabepilone Injection); Jakafi (Ruxolitinib); Jevtana (Cabazitaxel Injection); Kadcyla (Ado-trastuzumab Emtansine); Keytruda (Pembrolizurnab Injection); Kyprolis (Carfilzomib); Lanvima (Lenvatinib); Leukeran (Chlorambucil); Leukine (Sargramostim); Leustatin (Cladribine); Lonsurf (Trifluridine and Tipiracil); Lupron (Leuprolide); Lupron Depot (Leuprolide); Lupron DepotPED (Leuprolide); Lynparza (Olaparib); Lysodren (Mitotane); Marqibo Kit (Vincristine Lipid Complex Injection); Matulane (Procarbazine); Megace (Megestrol); Mekinist (Trametinib); Mesnex (Mesna); Mesnex (Mesna Injection); Metastron (Strontium-89 Chloride); Mexate (Methotrexate Injection); Mustargen (Mechlorethamine); Mutamycin (Mitomycin); Myleran (Busulfan); Mylotarg (Gemtuzumab Ozogamicin); Navelbine (Vinorelbine); Neosar Injection (Cyclophosphamide Injection); Neulasta (filgrastim); Neulasta (pegfilgrastim); Neupogen (filgrastim); Nexavar (Sorafenib); Nilandron (Nilandron (nilutamide)); Nipent (Pentostatin); Nolvadex (Tamoxifen); Novantrone (Mitoxantrone); Odomzo (Sonidegib); Oncaspar (Pegaspargase); Oncovin (Vincristine); Ontak (Denileukin Diftitox); Onxol (Paclitaxel Injection); Opdivo (Nivolumab Injection); Panretin (Alitretinoin); Paraplatin (Carboplatin); Perjeta (Pertuzumab Injection); Platinol (Cisplatin); Platinol (Cisplatin Injection); PlatinolAQ (Cisplatin); PlatinolAQ (Cisplatin Injection); Pomalyst (Pomalidomide); Prednisone intensol (Prednisone); Proleukin (Aldesleukin); Purinethol (Mercaptopurine); Reclast (Zoledronic acid); Revlimid (Lenalidomide); Rheumatrex (Methotrexate); Rituxan (Rituximab); RoferonA alfaa (Interferon alfa-2a); Rubex (Doxorubicin); Sandostatin (Octreotide); Sandostatin LAR Depot (Octreotide); Soltamox (Tamoxifen); Sprycel (Dasatinib); Sterapred (Prednisone); Sterapred DS (Prednisone); Stivarga (Regorafenib); Supprelin LA (Histrelin Implant); Sutent (Sunitinib); Sylatron (Peginterferon Alfa-2b injection (Sylatron)); Sylvant (Siltuximab Injection); Synribo (Omacetaxine Injection); Tabloid (Thioguanine); Taflinar (Dabrafenib); Tarceva (Erlotinib); Targretin Capsules (Bexarotene); Tasigna (Decarbazine); Taxol (Paclitaxel Injection); Taxotere (Docetaxel); Temodar (Temozolomide); Temodar (Temozolomide Injection); Tepadina (Thiotepa); Thalomid (Thalidomide); TheraCys BCG (BCG); Thioplex (Thiotepa); TICE BCG (BCG); Toposar (Etoposide Injection); Torisel (Temsirolimus); Treanda (Bendamustine hydrochloride); Trelstar (Triptorelin Injection); Trexall (Methotrexate); Trisenox (Arsenic trioxide); Tykerb (lapatinib); Unituxin (Dinutuximab Injection); Val star (Valrubicin Intravesical); Vantas (Histrelin Implant); Vectibix (Panitumumab); Velban (Vinblastine); Velcade (Bortezomib); Vepesid (Etoposide); Vepesid (Etoposide Injection); Vesanoid (Tretinoin); Vidaza (Azacitidine); Vincasar PFS (Vincristine); Vincrex (Vincristine); Votrient (Pazopanib); Vumon (Teniposide); Wellcovorin IV (Leucovorin Injection); Xalkori (Crizotinib); Xeloda (Capecitabine); Xtandi (Enzalutamide); Yervoy (Ipilimumab Injection); Yondelis (Trabectedin Injection); Zaltrap (Ziv-aflibercept Injection); Zanosar (Streptozocin); Zelboraf (Vemurafenib); Zevalin (Ibritumomab Tiuxetan); Zoladex (Goserelin); Zolinza (Vorinostat); Zometa (Zoledronic acid); Zortress (Everolimus); Zydelig (Idelalisib); and Zykadia (Ceritinib).

To examine mechanisms of resistance to immunotherapy, we performed genome-wide sequence analysis of protein coding genes as well as T cell receptor (TCR) clonotype analysis of patients that demonstrated initial response to immune checkpoint blockade but ultimately developed progressive disease. These analyses identified mutation-associated neoantigen candidates that were lost in the resistant tumors either through tumor cell elimination or chromosomal deletions, suggesting novel mechanisms for acquisition of resistance to immune checkpoint blockade.

Despite the compelling clinical efficacy of immune check point inhibitors, a subset of patients acquire resistance after an initial response to these therapies. We examined a variety of mechanisms that have been proposed in the development of resistance to immunotherapies¹⁸. Response to PD-1 blockade has been associated with PD-L1 protein expression and may play a role in therapeutic benefit and emergence of resistance^{8,19}. However, we did not observe any differences in PD-L1 expression in tumor cells between responsive and resistant tumor samples (Supplementary FIG. 10). Loss of antigen-presenting molecules might be an alternative mechanism of resistance to immune checkpoint blockade¹⁰). We did not observe any loss-of-function mutations in the HLA genes or the transporter for antigen presentation (TAP-1) gene in the resistance tumor specimens. Similarly, we did not identify any LOH events in the HLA class I and II and TAP-1 loci on chromosome 6 for any of the resistance tumor specimens. PTEN loss has been recently been shown to inhibit T-cell tumor infiltration and promotes resistance to PD-1 blockade in melanoma²⁰ but loss of function mutations in PTEN were not identified in resistant tumors we analyzed. Although we were precluded from evaluating other immune modulators due limited biopsy specimens, it is conceivable that transcriptomic signatures²¹ or specific co-inhibitory factors, such as T-cell immunoglobulin mucin-3 (TIM-3)⁹, may play a role in immune checkpoint regulation.

Through our comprehensive genomic analyses we showed that emergence of acquired resistance may be mediated by neoantigen loss through elimination of tumor subclones or chromosomal loss of truncal alterations. Acquisition of somatic resistance mutations is a common mechanism of therapeutic resistance to targeted therapies²². However, loss of somatic mutations through subsequent genetic events is uncommon in the context of natural tumor evolution or therapeutic resistance^{23,24}. In our resistant samples, the eliminated mutations were in genes that are typically expressed at higher levels in lung cancer and encoded for neoantigens that were predicted to have high affinity for MHC binding. It is conceivable that the identified neoantigens eliminated at the time of emergence of resistance were immunodominant²⁵. In this setting, the immune system becomes “addicted” to these neantigens, ignoring other tumor-related antigens, and after neoantigen loss during therapy the immune system cannot mount an effective anti-tumor response.

Deciphering the mechanisms through which cancer adapts to evade anti-tumor immune responses is critical for the development of tailored cancer immunotherapy strategies. Putative neoantigens identified prior to and at the time of emergence of resistance can be used to develop patient-specific immunotherapy approaches including vaccines, adoptive T-cell transfer or chimeric antigen receptor (CAR) T cell therapy. Our results suggest that immunotherapies targeting clonal neoantigens are likely to be more effective than those targeting subclonal neoantigens, as elimination of these changes may be more challenging for the tumor than subclonal alterations. These approaches may augment the efficacy of immunotherapy in patients that demonstrate initial responses but ultimately develop acquired resistance to immune checkpoint blockade.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLE 1

### Case Reports

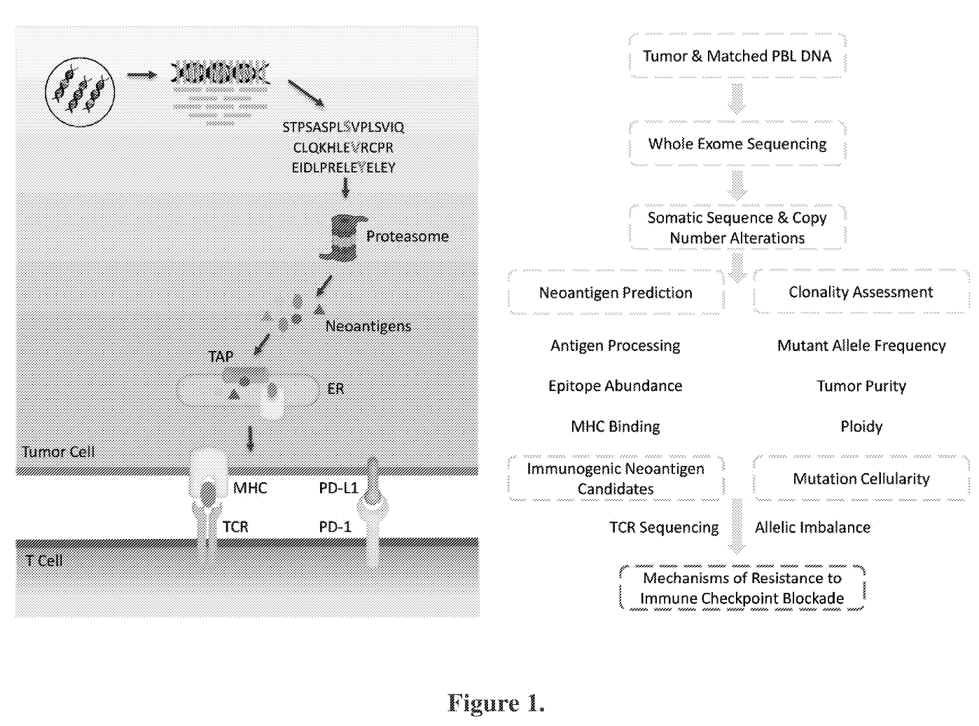

Patient CGLU116 was a 55-year-old male, 40 pack-year ex-smoker, initially diagnosed with stage IIB squamous lung cancer, treated with left pneumonectomy followed by adjuvant cisplatin, vinorelbine and bevacizumab. Upon disease recurrence he was enrolled on a clinical trial of concurrent anti-PD-1 and anti-CTLA4 therapy and achieved a partial response as defined by RECIST 1.1 criteria after one dose of combined treatment (Supplementary FIG. 1). Due to treatment-related toxicities and sustained response, he did not receive further anticancer therapy and developed progressive disease with left pleural implants 11 months later.

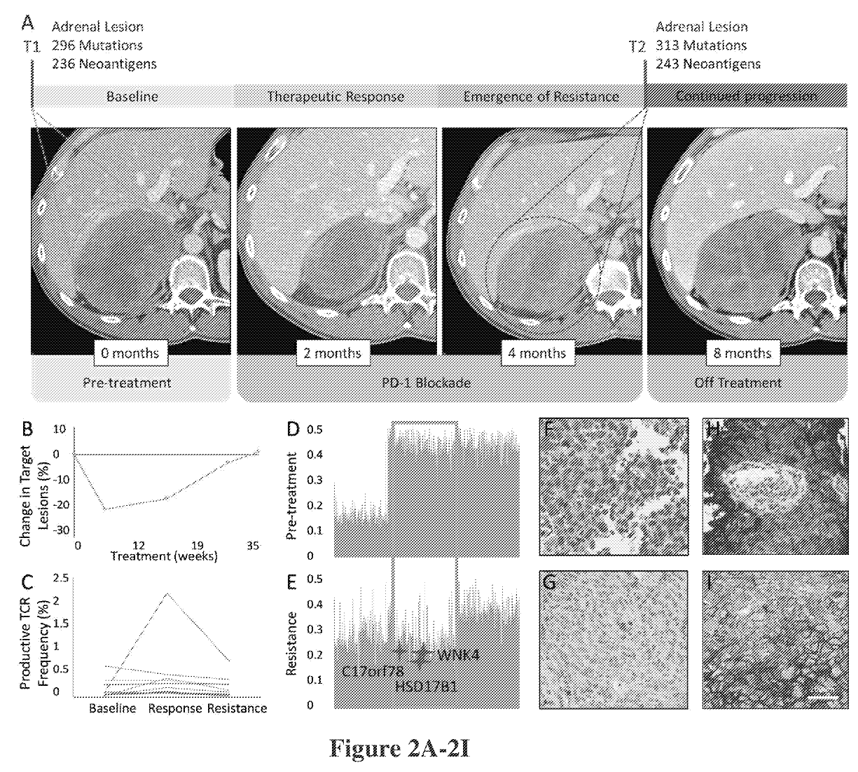

Patient CGLU117 was a 55-year-old male, 80 pack-year current smoker, diagnosed with stage IIIA EGFR/KRAS/ALK wild-type lung adenocarcinoma. Following progression in a solitary site (right adrenal metastasis) immediately after definitive chemo-radiation with cisplatin and etoposide, and continued progression on 1st line chemotherapy with carboplatin, pemetrexed and bevacizumab, he received anti-PD-1 therapy. He achieved stable disease (22% tumor regression by RECIST 1.1) of 4 months duration before he developed disease progression within the enlarging right adrenal metastasis (FIG. 2).

Patient CGLU127 was a 58-year-old female, 40 pack-year ex-smoker diagnosed with stage IV KRAS mutant (13G>C) lung adenocarcinoma, initially treated with carboplatin, paclitaxel and cetuximab, followed by second line pemetrexed. Upon disease progression she commenced anti-PD1 therapy and achieved a partial response for 6 months but subsequently relapsed with increased hilar, mediastinal and retroperitoneal nodal and pleural disease as well as left adrenal metastasis (Supplementary FIG. 2).

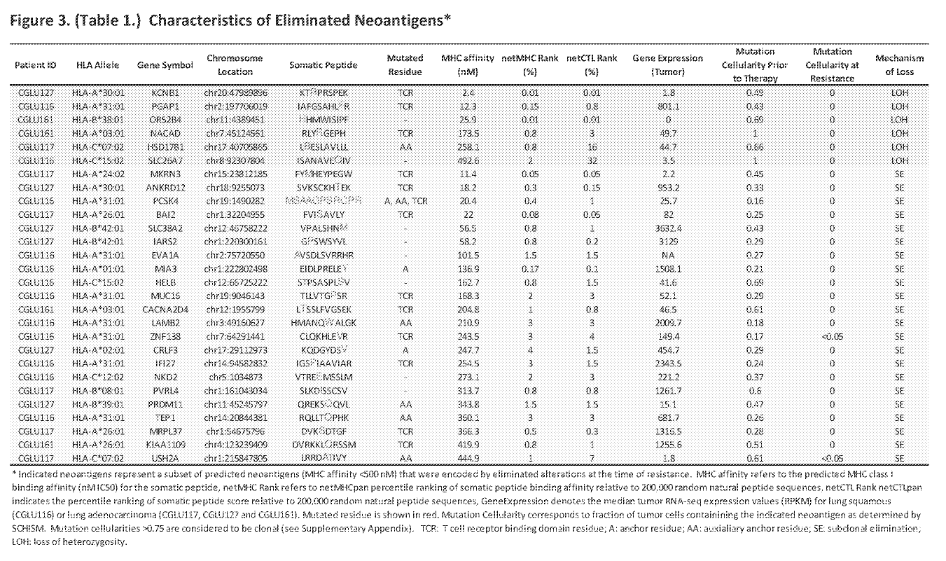

CGLU161 was a 42-year-old male, 5 pack-year distant ex-smoker, with history of mantle field radiation to the chest for Hodgkin Lymphoma at age 19, diagnosed with stage IV lung adenocarcinoma with liver metastasis. His tumor was wild type for EGFR, ALK and KRAS and he was enrolled in a 1st line clinical trial of combined PD-1 and CTLA4 blockade. He achieved a partial response of 7 months duration before disease progression with brain metastasis and diffuse tumor infiltration of the liver parenchyma (Supplementary FIG. 3).

Clinical and pathological characteristics for all patients are summarized in Supplementary Table 1 and tumor burden kinetics are shown in FIG. 2 and in the Supplementary Appendix. Whole exome sequencing, somatic mutation detection and neoantigen predictions, as well as PD-L1 and CD8 immunohistochemistry were performed on pre-treatment and post-progression tumor samples while comprehensive TCR clonotypes were assessed in pre-treatment and post-progression tumors and peripheral blood lymphocytes (PBLs).

### EXAMPLE 2

### Methods

### Patient and Sample Characteristics

Our study group consisted of 4 lung cancer patients treated with immune checkpoint blockade at Johns Hopkins Sidney Kimmel Cancer Center. The study was approved by the Institutional Review Board (IRB) and patients provided written informed consent for sample acquisition for research purposes.

### Whole-Exome Sequencing, Neoantigen Prediction and T Cell Receptor Sequencing

Whole exome sequencing was performed on the pre-treatment and post-progression tumor and matched normal samples. Tumor and normal sequence data were compared to identify somatic and germline alterations using the VariantDx software pipeline¹², focusing on single base substitutions as well as small insertions and deletions. To assess the immunogenicity of somatic mutations, exome data combined with each individual patient's major histocompatibility complex (MHC) class I haplotype were applied in a neoantigen prediction platform that evaluates binding of somatic peptides to class I MHC, antigen processing, self-similarity and gene expression. TCR clones were evaluated in pre-treatment and post progression tumor tissue and matching PBLs by next generation sequencing.

### Immunohistochemistry

Tumor sections were deparaffinized and stained with primary antibodies against PD-L1 and CD8 as described in the Supplementary Appendix.

### Analysis of Mutation Cellularity and Tumor Cell Clonality

The tumor subclonality phylogenetic reconstruction algorithm SCHISM 1.1¹³ was used to infer mutation cellularity in each patient using observed read counts and adjustments based on allelic imbalance and tumor purity.

### Statistical Analysis

Mann-Whitney U-test was employed to compare metrics of neoantigen binding and expression. All p values were based on 2-sided testing and differences were considered significant at p<0.05. Statistical analyses were performed with SPSS (version 22 for windows).

### EXAMPLE 3

### Patient and Sample Characteristics

Two patients (CGLU117 and CGLU127) were treated with single agent nivolumab between December 2014 and October 2015 (Institutional Review Board-IRB study number J1353) and 2 patients (CGLU116 and CGLU161) were treated with nivolumab and ipilimumab between July 2014 and October 2015 (IRB study number J11106). Patients underwent tumor biopsies within 30 days of starting treatment and at the time of progression, with the exception of CGLU116 for which an archival specimen from the time of the patient's pneumonectomy was analyzed as the baseline tumor sample. All tumor samples were provided as formalin fixed paraffin embedded blocks (FFPE). Four pre-treatment and five post-progression specimens (2 progression samples for patient CGLU161) and their matched normal tissues were obtained and analyzed with IRB approval and patients' consents. Serial blood samples were collected to assess immune responses: for patients CGLU117 and CGLU127, samples were obtained prior to treatment initiation, at the time of response to nivolumab and at the time of disease progression. For patient CGLU161 blood was collected at the time of response to nivolumab and ipilimumab and at the time of disease progression. Blood samples from the time of disease progression were available for patient 116. Clinical and pathological characteristics for all patients are summarized in Supplementary Table 1 (all supplementary tables and figures are available on-line at the publisher's website).

### Treatment and Assessment of Clinical Response

CGLU117 and CGLU127 received single agent nivolumab at 3 mg/kg every 2 weeks. CGLU116 and CGLU161 received nivolumab 1 mg/kg every 2 weeks and ipilimumab 1 mg/kg every 6 weeks. Tumor responses to immune checkpoint blockade were evaluated every 8 weeks after treatment initiation. The response evaluation criteria in solid tumors (RECIST) version 1.1 were used to determine clinical responses. Based on RECIST criteria patients CGLU116, CGLU117, CGLU161 had a partial response as best response and patient CGLU117 had stable disease (22% tumor regression). Patient CGLU116 achieved a deep partial response after one dose of nivolumab and ipilimumab however was not able to receive further treatment because of treatment-related toxicity. Computed tomographic findings and tumor burden kinetics are shown in FIG. 2 and Supplementary FIGS. 1-4.

### Sample Preparation and Next-Generation Sequencing

Tumor samples underwent pathological review for confirmation of lung cancer diagnosis and assessment of tumor cellularity. Slides from each FFPE block were macrodissected to remove contaminating normal tissue. Matched normal samples were provided as peripheral blood. DNA was extracted from patients' tumors and matched peripheral blood using the Qiagen DNA FFPE and Qiagen DNA blood mini kit respectively (Qiagen, CA). Briefly, tumor samples were incubated in proteinase K for 16-20 hours, followed by DNA fragmentation for 10 minutes in a Covaris sonicator (Covaris, Woburn, Mass.) to a size of 150-450 bp. Samples were further digested for 1 hour followed by incubation for an hour at 80° C. Fragmented genomic DNA from tumor and normal samples used for Illumina TruSeq library construction (Illumina, San Diego, Calif.) according to the manufacturer's instructions as previously described^{1,2}. DNA was mixed with 36 μl of H2O, 10 μl of End Repair Reaction Buffer, 5 μl of End Repair Enzyme Mix (cat# E6050, NEB, Ipswich, Mass.). The 100 μl end-repair mixture was incubated at 20° C. for 30 min, and purified using Agencourt AMPure XP beads (Beckman Coulter, IN) in a ratio of 1.0 to 1.25 of PCR product to beads and washed using 70% ethanol per the manufacturer's instructions. To A-tail, 42 μl of end-repaired DNA was mixed with 5 μl of 10×dA Tailing Reaction Buffer and 3 μl of Klenow (cat# E6053, NEB, Ipswich, Mass.). The 50 μl mixture was incubated at 37° C. for 30 min and purified using Agencourt AMPure XP beads (Beckman Coulter, IN) in a ratio of 1.0 to 1.0 of PCR product to beads and washed using 70% ethanol per the manufacturer's instructions. For adaptor ligation, 25 μl of A-tailed DNA was mixed with 6.7 pi of H2O, 3.3 μl of PE-adaptor (Illumina), 10 μl of 5× Ligation buffer and 5 μl of Quick T4 DNA ligase (cat# E6056, NEB, Ipswich, Mass.). The ligation mixture was incubated at 20° C. for 15 min and purified using Agencourt AMPure XP beads (Beckman Coulter, IN) in a ratio of 1.0 to 0.95 and 1.0 of PCR product to beads twice and washed using 70% ethanol per the manufacturer's instructions. To obtain an amplified library, twelve PCRs of 25 μl each were set up, each including 15.5 μl of H2O, 5 μl of 5× Phusion HF buffer, 0.5 μl of a dNTP mix containing 10 mM of each dNTP, 1.25 μl of DMSO, 0.25 μl of Illumina PE primer #1, 0.25 μl of Illumina PE primer #2, 0.25 μl of Hotstart Phusion polymerase, and 2 μl of the DNA. The PCR program used was: 98° C. for 2 minutes; 12 cycles of 98° C. for 15 seconds, 65° C. for 30 seconds, 72° C. for 30 seconds; and 72° C. for 5 min. DNA was purified using Agencourt AMPure XP beads (Beckman Coulter, IN) in a ratio of 1.0 to 1.0 of PCR product to beads and washed using 70% ethanol per the manufacturer's instructions. Exonic regions were captured in solution using the Agilent SureSelect v.4 kit according to the manufacturer's instructions (Agilent, Santa Clara, Calif.). The captured library was then purified with a Qiagen MinElute column purification kit and eluted in 17 μl of 70° C. EB to obtain 15 μl of captured DNA library. The captured DNA library was amplified in the following way: eight 30 uL PCR reactions each containing 19 μl of H2O, 6 μl of 5× Phusion HF buffer, 0.6 μl of 10 mM dNTP, 1.5 μl of DMSO, 0.30 μl of Illumina PE primer #1, 0.30 μl of Illumina PE primer #2, 0.30 μl of Hotstart Phusion polymerase, and 2 μl of captured exome library were set up. The PCR program used was: 98° C. for 30 seconds; 14 cycles of 98° C. for 10 seconds, 65° C. for 30 seconds, 72° C. for 30 seconds; and 72° C. for 5 min. To purify PCR products, a NucleoSpin Extract II purification kit (Macherey-Nagel, PA) was used following the manufacturer's instructions. Paired-end sequencing, resulting in 100 bases from each end of the fragments for the exome libraries was performed using Illumina HiSeq 2000/2500 instrumentation (Illumina, San Diego, Calif.).

### Primary Processing of Next-Generation Sequencing Data and Identification of Putative Somatic Mutations

Somatic mutations were identified using VariantDx custom software for identifying mutations in matched tumor and normal samples². Prior to mutation calling, primary processing of sequence data for both tumor and normal samples were performed using Illumina CASAVA software (version 1.8), including masking of adapter sequences. Sequence reads were aligned against the human reference genome (version hg19) using ELAND with additional realignment of select regions using the Needleman-Wunsch method3. Candidate somatic mutations, consisting of point mutations, insertions, deletions as well as copy number changes were then identified using

VariantDx across the whole exome. VariantDx examines sequence alignments of tumor samples against a matched normal while applying filters to exclude alignment and sequencing artifacts. In brief, an alignment filter was applied to exclude quality failed reads, unpaired reads, and poorly mapped reads in the tumor. A base quality filter was applied to limit inclusion of bases with reported Phred quality score >30 for the tumor and >20 for the normal. A mutation in the pre or post treatment tumor samples was identified as a candidate somatic mutation only when (1) distinct paired reads contained the mutation in the tumor; (2) the fraction of distinct paired reads containing a particular mutation in the tumor was at least 10% of the total distinct read pairs and (3) the mismatched base was not present in >1% of the reads in the matched normal sample as well as not present in a custom database of common germline variants derived from dbSNP and (4) the position was covered in both the tumor and normal. Mutations arising from misplaced genome alignments, including paralogous sequences, were identified and excluded by searching the reference genome. Alterations in cases where both tumor samples had tumor purity <50% (CGLU116) were analyzed with the above criteria except that the minimum fraction of distinct reads was 5%. For case CGLU161 where two tumor samples were available after initiation of therapy, shared alterations were those that were present in T1, T2 and T3, or T1 and T2 or T1 and T3, while those that were considered lost could be absent in either T2 or T3.

Candidate somatic mutations were further filtered based on gene annotation to identify those occurring in protein coding regions. Functional consequences were predicted using snpEff and a custom database of CCDS, RefSeq and Ensembl annotations using the latest transcript versions available on hg19 from UCSC (see the genome website of the University of Sourthern California). Predictions were ordered to prefer transcripts with canonical start and stop codons and CCDS or Refseq transcripts over Ensembl when available. Finally mutations were filtered to exclude intronic and silent changes, while retaining mutations resulting in missense mutations, nonsense mutations, frameshifts, or splice site alterations. A manual visual inspection step was used to further remove artefactual changes. An analysis of each candidate mutated region either gained or lost in post-progression specimens was performed using BLAT. For each mutation, 101 bases including 50 bases 5′ and 3′ flanking the mutated base was used as query sequence (http://genome.ucsc.edu/cgi-bin/hgBlat).

Candidate mutations were removed from further analysis, if the analyzed region resulted in >1 BLAT hits with 90% identity over 90 SPAN sequence length.

### Neoantigen Predictions

Detected somatic mutations, consisting of non-synonymous single base substitutions, insertions and deletions, were evaluated for putative neoantigens using the ImmunoSelect-R pipeline (Personal Genome Diagnostics, Baltimore, Md.). Briefly, ImmunoSelect-R performs a comprehensive assessment of paired somatic and wild type peptides 8-11 amino acids in length at every position surrounding a somatic mutation. To accurately infer a patient's germline HLA 4-digit allele genotype, whole-exome-sequencing data from paired tumor/normal samples were first aligned to a reference allele set, which was then formulated as an integer linear programming optimization procedure to generate a final genotype⁴. The HLA genotype served as input to netMHCpan to predict the MHC class I binding potential of each somatic and wild-type peptide (IC50 nM), with each peptide classified as a strong binder (SB), weak binder (WB) or non-binder (NB)⁵⁻⁷. Peptides were further evaluated for antigen processing (neCTLpan⁸) and were classified as cytotoxic T lymphocyte epitopes (E) or non-epitopes (NA). Paired somatic and wild-type peptides were assessed for self-similarity based on MHC class binding affinity⁹. Neoantigen candidates meeting an IC50 affinity <5000 nM were subsequently ranked based on MHC binding and T-cell epitope classifications. Tumor-associated expression levels derived from TCCA were used to generate a final ranking of candidate immunogenic peptides. Anchor and auxiliary anchor residues for mutant peptides-HLA class I allele pairs were evaluated by the SYFPEITHI online tool (www.syfpeithi.de)¹⁰. To generate Table 1 we filtered the neoantigen predictions by applying a 500 nM affinity threshold and reduced the redundancy by selecting the strongest binding neoepitope specific to an HLA allele with known binding motifs in SYFPEITHI.

### Somatic Copy Number Analysis

Genome-wide copy number profile of each tumor sample was derived by comparing the abundance of aligned reads to each region between tumor and matched normal samples using the CNVKit method11. CNVkit enables inference and visualization of copy number aberrations from sequencing data. The method uses sequencing reads mapped to the exome, as well as non-specifically captured reads, and corrects the sequencing depth profile with respect to three sources of bias: GC-content, capture target size, and regions containing sequence repeats. We derived a preliminary estimate of genome-wide copy number profile of each tumor sample as quantified by log2 ratio of reads between tumor and matched normal. Next we estimated the tumor purity by cross-analysis of these log2 ratio values and minor allele frequency of germline heterozygous variants. The estimated tumor purity (p) was used to convert the observed raw log2 ratio (r) to tumor copy number (CN_{T}) correcting for contribution of normal cell copy number (CN_{N}) as follows:

r=log₂((CN_{T}*p+CN_{N}*(1−p))/CN)

The corresponding tumor copy number values were rounded to closest integer levels to yield the final somatic copy number profile.

### Tumor Purity Estimation

Normal cell contamination is one of the factors complicating the analysis of somatic alterations in solid tumors¹². To estimate the purity of each tumor sample, we extended the framework of SCHISM 1.1.1. to cross-analyze the preliminary somatic copy number profile, and the minor allele frequency distribution of germline heterozygous single nucleotide polymorphisms (SNPs) along the genome. In each tumor sample, we selected a candidate subset of chromosomes or chromosome arms where there was a clear deviation of the minor allele frequencies from the expected value of 0.5, and log2 ratio of read counts indicated one copy loss by visual inspection. The expected minor allele frequency of germline heterozygous SNPs was calculated as

mafₗₒₛₛ=[n_{T}^{m}*p+nₘ^{m}*(1−p)]/[CN_{T}*p+CN_{N}*(1−p)]

where p is the proportion of cancer cells in the sequenced tumor bulk (tumor purity), and n_{T}^{m} and n_{N}^{m} are the number of copies of minor allele present in tumor and normal cells, respectively. In regions of one copy loss, the minor allele is absent in tumor cells (n_{T}^{m}=0) and present in one copy in normal cells (n_{N}^{m}=1), tumor copy number is one (CN_{T}=1) and normal copy number is two (CN_{N}=2), therefore:

mafₗₒₛₛ=(1−p)/(2−p)

We identified the mode of minor allele frequency in each such region and estimated the tumor purity as the average purity values estimated for the analyzed regions.

### Genome-Wide Analysis of Allelic Imbalance

In each tumor sample, we examined evidence for allelic imbalance in genomic regions surrounding somatic mutations. For each mutation, we compared the minor allele frequency of 20 closest germline heterozygous SNPs with coverage of at least 10 reads between tumor and matched normal sample using a 1-sided t-test. The p-values were corrected for multiple hypothesis testing using Benjamini-Hochberg¹³ procedure. Regions with false discovery rate (FDR) less than or equal to 0.05 and a difference of at least 0.10 between the average minor allele frequencies of tumor and normal were marked as harboring allelic imbalance.

### Somatic Mutation Cellularity Estimation

Estimating the fraction of cancer cells harboring each somatic mutation (mutation cellularity) is central to reconstruction of subclone hierarchies and tumor evolution. We used an extension of the framework in SCHISM-1.1.114 to derive point estimates and confidence intervals of mutation cellularities as follows: For each mutation, the expected value of variant allele frequency Vexp was determined by tumor sample purity p, tumor copy number CN_{T}, normal copy number CN_{N}, mutation cellularity C and mutation multiplicity m. Mutation multiplicity refers the number of mutant alleles present in tumor cells harboring the mutation. The expected variant allele frequency was calculated as:

Vexp=mCp/[pCN_{T}+(1−p)CN_{N}]

For each mutation, we derived a cellularity estimate at each possible multiplicity values (in absence of allele specific tumor copy number, ∈{1, . . . , CN_{T}}) as follows. Given a multiplicity value m, we found the expected variant allele frequency for each value of cellularity in Cg{0.00, 0.01, . . . , 1.00}. Next, we found the binomial likelihood of observing r_{B} variant reads out of r_{T} total reads covering the mutation where success probability is set to Vexp. We normalized these likelihood values to sum to one, and derived the maximum likelihood estimate of cellularity and the 95% confidence interval using this normalized likelihood distribution over Cg.

We selected the level of multiplicity for each mutation in each sample as follows: The multiplicity for imitations with tumor copy number of 1 is 1. Mutations with tumor copy number of 2 and outside regions with allelic imbalance are assumed to have multiplicity of 1. Mutations with tumor copy number of 2, and in regions with allelic imbalance are assumed to have multiplicity of 2. For mutations that are lost where allelic imbalance was absent in pre-treatment sample and was present in post-treatment sample, multiplicity is assumed to be 1. Mutations absent in pre-treatment sample and in regions with constant tumor copy number between pre- and post-treatment samples have multiplicity of 1. Finally, for mutations lost where tumor copy number changes from 3 in pre-treatment to 2 in post-treatment sample, and allelic imbalance only present in pre-treatment sample, multiplicity is assigned to 1. For mutations where multiplicity (and cellularity) could not be determined using the above approach, we used a secondary method. This involves clustering above mutations to identify groups of mutations with similar cellularity across all available samples. For each unclassified mutation, the unresolved cellularity (and multiplicity) values are selected to minimize the distance to the closest mutation cluster. A cellularity >0.75 was used to differentiate truncal from subclonal mutations.

### T Cell Receptor Sequencing

DNA from pre- and post-treatment tumor samples and peripheral blood lymphocytes (PBLs) was isolated by using the Qiagen DNA FFPE and Qiagen DNA blood mini kit respectively (Qiagen, CA). TCR-β CDR3 regions were amplified using the survey (tumor) or deep (PBLs) ImmunoSeq assay in a multiplex PCR method using 45 forward primers specific to TCR Vβ gene segments and 13 reverse primers specific to TCR Jβ gene segments (Adaptive Biotechnologies)^{15,16}. Productive TCR sequences were further analyzed. The top 100 most frequent TCR clones in the tumor were used to determine their frequencies in peripheral blood prior to treatment, at the time of response and upon emergence of resistance. For each sample, a clonality metric was estimated in order to quantitate the extent of mono- or oligo-clonal expansion by measuring the shape of the clone frequency distribution¹⁹. Clonality values range from 0 to 1 where values approaching 1 indicate a nearly monoclonal population (Supplementary Table 10).

### Immunohistochemistry and Interpretation of PD-L1 and CD8 Staining

Immunohistochemistry for PD-LI was performed using the PD-L1 IHC 22C3 pharmDx assay kit (Dako, Calif.). In brief, slides were deparaffinized with xylene and rehydrated with ethanol. Antigen retrieval was performed using citrate buffer (pH=6) at a temperature of 97° C. for 20 min. After blocking of endogenous peroxidase, slides were incubated with the primary mouse anti-human PD-L1 antibody (clone 22C3) or the negative control reagent for 30 min at room temperature. Slides were then incubated with an anti-mouse Linker antibody, followed by a 30 minute incubation with the FLEX+ secondary antibody/horseradish peroxidase polymer system. Signal was visualized with 3,3′ diaminobenzidine (DAB) and slides were counterstained with hematoxylin and coverslipped. NCI-226, a lung cancer cell line with known PD-L1 protein expression, and MCF-7, a breast cancer cell line with negative PD-L1 protein expression were used as positive and negative controls respectively. Negative control sections, in which the primary antibody was omitted were also used for each immunostaining run. A minimum of 100 tumor cells were evaluated per specimen; only membranous staining was considered specific and further interpreted. PD-L1 protein expression was evaluated based on the intensity of staining on a 0 to 3+ scale, and the percentage of immune-reactive tumor cells. Samples with membranous PD-L1 staining with an intensity score of 2+ in at least 1% of cells were classified as PD-L1 positive. Similarly, slides were deparaffinized, rehydrated, antigen retrieved and incubated with a mouse anti-human CD8 antibody (Dako, Calif.) diluted 1:100 overnight at 4° C., followed by a 30 minute incubation with the FLEX+ polymer system. DAB was used for signal visualization, sections were subsequently counterstained with hematoxylin and coverslipped. CD8-positive lymphocyte density was evaluated per 20× high power field. CD8 expression was evaluated in pre-treatment and post-progression tissue specimens for CLGU117 (FIG. 2) and in post-progression specimens for CGLU 16 and CGLU161 (Supplementary FIG. 11) given limited. tissue availability for the remaining cases.

### Statistical Analysis

Somatic mutations found to harbor at least one candidate neoantigen were utilized to compare features of immunogenicity between those eliminated and those shared or gained after treatment across the four patients. Given a specific binding threshold (IC50), mutations that generated neoantigens were characterized for features including minimum predicted IC50 average predicted affinity, the number of strong binder classifications and corresponding gene expression. To reduce redundancy, somatic mutations with multiple peptides satisfying the IC50 threshold were represented by their average value for downstream statistical comparisons of lost and shared/gained groups. The unpaired Mann-Whitney U test was applied to compare lost and sharedlgained groups.

References for Example 3 only.

1. Sausen M, Leary R J, Jones S, et al. Integrated genomic analyses identify ARID1A and ARID1 B alterations in the childhood cancer neuroblastoma. Nature genetics 2013;45:12-7.

2. Jones S, Anagnostou V, Lytle K, et al. Personalized genomic analyses for cancer mutation discovery and interpretation. Science translational medicine 2015;7:283ra53.

3. Needleman S B, Wunsch C D. A general method applicable to the search similarities in the amino acid sequence of two proteins. J Mol Biol 1970;48:443-53.

4. Szolek A, Schubert B, Mohr C, Sturm M, Feldhahn M, Kohlbacher O. OptiType: precision HLA typing from next-generation sequencing data. Bioinformatics 2014;30:3310-6.

5. Nielsen M, Andreatta M. NetMHCpan-3.0; improved prediction of binding to MHC class I molecules integrating information from multiple receptor and peptide length datasets. Genome Med 2016;8:33.

6. Lundegaard C, Lamberth K, Harndahl M, Buus 5, Lund O, Nielsen M. NetMHC-3.0: accurate web accessible predictions of human, mouse and monkey MHC class I affinities for peptides of length 841. Nucleic Acids Res 2008;36:W509-12.

7. Lundegaard C, Lund O, Nielsen M. Accurate approximation method for prediction of class I MHC affinities for peptides of length 8, 10 and 11 using prediction tools trained on 9mers. Bioinformatics 2008;24:1397-8.

8. Stranzl T, Larsen M V, Lundegaard C, Nielsen M. NetCTLpan: pan-specific MHC class I pathway epitope predictions. Immunogenetics 2010;62:357-68.

9. Kim Y, Sidney J, Pinilla C, Sette A, Peters B. Derivation of an amino acid similarity matrix for peptide: MHC binding and its application as a Bayesian prior. BMC Bioinformatics 2009;10:394.

10. Rammensee H, Bachmann J, Emmerich N P, Bachor O A, Stevanovic S. SYFPEITHI: database for MHC ligands and peptide motifs. Immunogenetics 1999;50:213-9.

11. Talevich E, Shain, A. H., Botton, T., & Bastian, B. C. CNV it: Copy number detection and visualization for targeted sequencing using off-target reads. bioRxiv doi: http://dxdoiorg/101101/010876 2014.

12. Aran D, Sirota M, Butte A J. Systematic pan-cancer analysis of tumour purity. Nature communications 2015:6:8971.

13. Benjamini Y, & Hochberg, Y. Controlling the False Discovery Rate: A Practical and Powerful Approach to Multiple Testing. Journal of the Royal Statistical Society Series B (methodological) 1995;57:289-300.

14. Niknafs N, Beleva-Guthrie V, Naiman D Q, Karchin R. SubClonal Hierarchy Inference from Somatic Mutations: Automatic Reconstruction of Cancer Evolutionary Trees from Multi-region Next Generation Sequencing. PLoS Comput Biol 2015;11:e1004416.

15. Carlson C S, Emerson R O, Sherwood A M, et al. Using synthetic templateo design an unbiased multiplex PCR assay. Nature communications 2013;4:2680.

16. Robins H S, Campregher P V, Srivastava S K, et al. Comprehensive assessment of T-cell receptor beta-chain diversity in alphabeta T cells. Blood 2009;114:4099-107

### EXAMPLE 4

### Identification of Neoantigens Lost in Acquired Resistance

To examine the landscape of genomic alterations and associated neoantigens in these patients, we performed whole exome sequencing of nine samples from four NSCLC patients treated with single agent PD-1 or combined PD-1 and CTLA4 blockade (FIG. 1, Supplementary Table 1). In all cases we examined samples obtained prior to therapy as well as biopsies acquired at the time of resistance. For patient CGLU116 we analyzed the lung tumor from the time of diagnosis and an enlarging pleural nodule at the time of progression. For patient CGLU117 a solitary adrenal metastasis present prior to initiation of PD-1 blockade was analyzed and compared to the same, post-progression enlarging adrenal mass. For patient CGLU127 we studied the lung tumor prior to treatment and a hilar lymph node metastasis at the time of progression. For patient CGLU161 a mediastinal lymph node obtained prior to immunotherapy was analyzed whereas liver and brain metastatic lesions were analyzed at the time of therapeutic resistance. We used next-generation sequencing to examine the entire exomes of these tumors and matched normal specimens (FIG. 1). The mean depth of coverage for the pre-treatment and resistant tumors was 214× and 217× respectively, allowing us to identify sequence alterations and copy number changes in >20,000 genes (Supplementary Table 1).

We used a high-sensitivity mutation detection pipeline12 to identify 123, 296, 335 and 106 somatic sequence alterations in pre-immunotherapy tumor samples from patients CGLU116, CGLU117, CGLU127 and CGLU161, respectively. The number and type of alterations as well as specific driver genes identified, including TP53, KRAS, MYC, ARID1A, RB1, and SMARCA4 genes, were consistent with previous observations of sequence and copy number changes in NSCLC^{14,15} (Supplementary Tables 2, 3). Post-progression tumor samples revealed an increase in the number of overall somatic sequence changes, including 172, 313 and 354 somatic sequence alterations for CGLU116, CGLU117 and CGLU127, respectively. For patient CGLU 161 two tumor samples were analyzed from the time of disease progression, a liver metastasis that contained 113 changes and a brain metastasis that contained 170 mutations (Supplementary Tables 2, 3).

We examined the immunogenicity of proteins affected by somatic alterations using a multi-dimensional neoantigen prediction platform (see Supplementary Appendix). This approach allowed for identification of peptides within mutated genes that were predicted to be processed and presented by MHC class I proteins and therefore most likely to elicit an immune response. The algorithm evaluated the binding of mutant peptides (8-11mers) for patient-specific HLA class I alleles and ranked the neoantigens according to MHC binding affinity, antigen processing, and self-similarity. We also evaluated the average expression of altered genes in TCGA lung cancer specimens. We identified 102, 236, 305 and 88 alterations encoding neoantigens for pre-treatment tumors for CGLU116, CGLU117, CGLU127 and CGLU161, respectively. At the time of resistance to immune checkpoint blockade neoantigens corresponding to 140, 243 and 315 mutated genes were identified from tumors of patients CGLU116, CGLU117 and CGLU127 (Supplementary Table 4). Additionally, 93 and 142 neoantigens were identified in the liver and brain metastases of patient CGLU161 respectively.

### XAMPLE 5

### Mechanism of Acquired Resistance to Checkpoint Blockade

We evaluated the alterations observed in the tumor samples to see if they may provide insight into potential mechanisms of immunotherapy resistance. Samples analyzed at the time of resistance to checkpoint blockade contained new neoantigens that were not detected in the original tumor. However, there were no mutations or copy number changes in either pre-or post-therapy samples in the CD274 gene encoding for PD-L1, PDCD1 encoding for PD-1 or CTLA4 gene. Similarly, we did not identify any genomic alterations in HLA genes or other antigen presentation associated genes.

Interestingly, we observed that a subset of neoantigens present in the original tumors were eliminated in tumors resistant to checkpoint blockade (Table 1). This included 18, 11, 7, and 13 neoantigens that were not present in patients CGLU116, CGLU117, CGLU127 and CGLU161, respectively. All eliminated neoantigens stemmed from single-base substitutions with the exception of neopeptides generated by a frameshift mutation in PCSK4 for CGLU116. Among the neoantigens with MHC binding affinity <50 nM, the eliminated neoantigens had higher predicted MHC binding affinity than those present in the resistant tumors (14.5 nM for lost neoantigens vs 23.4 nM for retained neoantigens, p<0.05). Additionally, analysis of TCGA expression data showed that eliminated neoantigens were present in genes that were typically expressed at higher levels than genes containing neoantigens that were retained (1084.21 versus 594.02 RPKM, p<0.05). The mutations in 13 eliminated neoantigens were found in positions proximal to the complementarity determining regions of the TCR¹⁶, and are likely to be important for recognition of the mutant peptide especially when the wild type peptide is also presented¹⁷. A quarter of mutant peptides harbored mutations in either anchor or auxiliary anchor residues, presumably affecting MHC binding of these neoantigens (FIG. 3 (Table 1), Supplementary Appendix). Overall, these observations are consistent with the notion that the eliminated neoantigens were important for the achievement of initial therapeutic response to checkpoint blockade.

### EXAMPLE 6

### Mechanisms of Loss of Neoantigens

Conceptually, there could be two mechanisms of neoantigen loss in resistant tumors. The first is through the immune elimination of neoantigen-containing tumor cells that represent a subset of the tumor cell population, followed by subsequent outgrowth of the remaining cells. The second is through the acquisition of one or more genetic events in a tumor cell that results in neoantigen loss, followed by selection and expansion of the resistant clone. The first mechanism would only be possible for heterogeneous neoantigens while the second could serve as a mechanism of resistance for both clonal and subclonal alterations. To evaluate the contribution of these mechanisms to the loss of neoantigens, we analyzed the tumors both before and after therapy using the SCHISM pipeline¹³ and incorporating mutation frequency, tumor purity, and copy number variation to infer the fraction of cells containing a specific mutation (mutation cellularity) (see Supplementary Appendix). Through these approaches alterations with a mutation cellularity >0.75 were estimated to be present in all tumor cells (truncal) while the remainder were considered to be subclonal. Consistent with our predictions, we observed loss of 4 truncal and 45 subclonal neoantigens at the time of emergence of resistance (Supplementary Tables 5-9 and subset indicated in Table 1). Analysis of genome-wide structural alterations revealed that all clonal neoantigens were lost through genetic events involving chromosomal deletions and loss of heterozygosity (LOH) (FIG. 2, Supplementary FIGS. 5-8, Supplementary Tables 5-9). Subclonal neoantigens were lost either by LOH or through elimination of tumor subclones.

### EXAMPLE 7

### Clonality of Cytotoxic TCR Clonotypes and Acquired Resistance

We hypothesized that loss of neoantigens would lead to a decrease in clonality of cytotoxic TCR clonotypes, thus resulting in tumor immune evasion at the time of emergence of resistance. We analyzed serially collected PBLs, prior to immunotherapy initiation, at the time of clinical response, and at resistance for patients CGLU117 and CGLU127 and at the time of response and disease progression for patient CGLU161 (Supplementary Table 10). For patients CGLU127 and CGLU 117, we observed peripheral T cell expansion of a subset of the top 100 most frequent intratumoral clones, with the most frequent clones reaching a 44- and 25-fold increase in abundance in the blood at the time of response, respectively (FIG. 2 and Supplementary FIG. 9). Overall, oligoclonal T cell expansion peaked at the time of response while clonality decreased to baseline levels at the time of resistance. For patient CGLU117, this observation was consistent with the fact that CD8+ immune density did not change in pre-treatment and resistant tumors (FIG. 2). A similar decrease in abundance was observed for the predominant peripheral TCR clonotypes that were also present in the tumor upon acquisition of resistance for patient CGLU161 (Supplementary FIG. 9). Taken together, neoantigen loss in resistant tumors was associated with reversal of TCR clone expansion, suggesting that neoantigen elimination may shape cytotoxic T lymphocyte responses during checkpoint blockade.

### REFERENCES

The disclosure of each reference cited is expressly incorporated herein.

1. Voge stein B, Papadopoulos N, Velculescu V E, Zhou S, Diaz L A, Jr., Kinzler K W. Cancer genome landscapes. Science 2013;339:1546-58.

2. Schumacher T N, Schreiber R D. Neoantigens in cancer immunotherapy. Science 2015;348:69-74.

3. Rizvi N A, Hellmann M D, Snyder A, et al. Cancer immunology. Mutational landscape determines sensitivity to PD-1 blockade in non-small cell lung cancer. Science 2015;348:124-8.

4. Snyder A, Makarov V, Merghoub T, et al. Genetic basis for clinical response to CTLA-4 blockade in melanoma. The New England journal of medicine 2014;371:2189-99.

5. Van Allen E M, Miao D, Schilling B, et al. Genomic correlates of response to CTLA4 blockade in metastatic melanoma. Science 2015.

6. Tumeh P C, Harview C L, Yearley J H, et al. PD-1 blockade induces responses by inhibiting adaptive immune resistance. Nature 2014;515:568-71.

7. Herbst R S, Soria J C, Kowanetz M, et al. Predictive correlates of response to the anti-PD-L1 antibody MPDL3280A in cancer patients. Nature 2014;515:563-7.

8. Garan E B, Rizvi N A, Hui R, et al. Pembrolizumab for die treatment of non-small-cell lung cancer. The New England journal of medicine 2015;372:2018-28.

9. Koyama S, Akbay E A, Li Y Y, et al. Adaptive resistance to therapeutic PD-1 blockade is associated with upregulation of alternative immune checkpoints. Nature communications 2016;7:10501.

10. Maeurer M J, Gollin S M, Storkus W J, et al. Tumor escape from immune recognition: loss of HLA-A2 melanoma cell surface expression is associated with a complex rearrangement of the short arm of chromosome 6. Clinical cancer research: an official journal of the American Association for Cancer Research 1996;2:641-52.

11. Shukla S A, Rooney M S, Rajasagi M, et al. Comprehensive analysis of cancer-associated somatic mutations in class I HLA genes. Nat Biotechnol 2015;33:1152-8.

12. Jones S, Anagnostou V, Lytle K, et al. Personalized genomic analyses for cancer mutation discovery and interpretation. Science translational medicine 2015;7:283ra53.

13. Niknafs N, Beleva-Guthrie V, Naiman D Q, Karchin R. SubClonal Hierarchy Inference from Somatic Mutations: Automatic Reconstruction of Cancer Evolutionary Trees from Multi-region Next Generation Sequencing. PLoS Comput Biol 2015;11:e1004416.

14. Cann K L, Dellaire G. Heterochromatin and the DNA damage response: the need to relax. Biochem Cell Biol 2011;89:45-60.

15. Cancer Genome Atlas Research N. Comprehensive genomic characterization of squamous cell lung cancers. Nature 2012;489:519-25.

16. Rudolph M G, Stanfield R L, Wilson I A. How TCRs bind MHCs, peptides, and coreceptors. Annual review of immunology 2006;24:419-66.

17. Yadav M, Jhunjhunwala S, Phung Q T, et al. Predicting immunogenic tumour mutations by combining mass spectrometry and exome sequencing. Nature 2014;515:572-6.

18. Ribas A. Adaptive mine Resistance: How Cancer Protects from m Attack. Cancer discovery 2015;5:915-9.

19. Topalian S L, Taube J M, Anders R A, Pardoll D M. Mechanism-driven biomarkers to guide immune checkpoint blockade in cancer therapy. Nature reviews 2016;16:275-87.

20. Peng W, Chen J Q, Liu C, et al. Loss of PTEN Promotes Resistance to T Cell-Mediated Immunotherapy. Cancer discovery 2016;6:202-16.

21. Hugo W, Zaretsky J M, Sun L, et al. Genomic and Transcriptomic Features of Response to Anti-PD-1 Therapy in Metastatic Melanoma. Cell 2016;165:35-44.

Bertotti A, Papp E, Jones S, et al. The genomic landscape of response to EGFR blockade in colorectal cancer. Nature 2015;526:263-7.

23. Jones S, Chen W D, Parmigiani G, et al. Comparative lesion sequencing provides insights into tumor evolution. Proceedings of the National Academy of Sciences of the United States of America 2008;105:4283-8.

24. Yachida S Jones S, Bozic I, et al. Distant metastasis occurs late during the genetic evolution of pancreatic cancer. Nature 2010;467:1114-7.

25. Dudley M E, Roopenian D C. Loss of a unique tumor antigen by cytotoxic T lymphocyte immunoselection from a 3-methylcholanthrene-induced mouse sarcoma reveals secondary unique and shared antigens. The Journal of experimental medicine 1996;184:441-7.

We claim:

1. A method of identifying target epitopes for a tumor of an individual, comprising:
performing massively parallel sequencing on a first sample of the individual comprising tumor DNA, on a second sample from the individual comprising normal tissue DNA, and on a third sample from the individual comprising tumor DNA, wherein the first sample is obtained prior to treatment with an anti-tumor agent and the third sample is obtained after treatment with the anti-tumor agent;
identifying somatic mutations in the first sample that encode a different amino acid sequence than in the second sample and form mutant epitopes;
analyzing the mutant epitopes in the first sample to identify epitopes that are recognized by class I MHC molecules of a type expressed by the individual; and
identifying from among the epitopes that are recognized by class I MHC molecules of the type expressed by the individual a first particular mutant epitope that is absent in the third sample and a second particular mutant epitope that is present in the third sample.

2. The method of claim 1 further comprising testing a sample of the patient to determine class I MHC alleles carried by the patient.

3. The method of claim 1 further comprising testing the mutant epitopes by contacting them with class I MHC molecules of the type expressed by the individual.

4. The method of claim 1 further comprising the step of testing the first sample to determine expression level of proteins in the tumor that comprise the mutant epitopes that are recognized by class I MHC molecules of the type expressed by the individual.

5. The method of claim 1 further comprising the step of testing the first sample to determine expression level of RNA in the tumor that encodes the mutant epitopes that are recognized by class I MHC molecules of the type expressed by the individual.

6. The method of claim 1 further comprising the step of determining affinity of the class I MHC molecule for the mutant epitope epitopes that are recognized by class I MHC molecules of the type expressed by the individual.

7. The method of claim 1 further comprising the step of testing one or more first samples and determining fraction of cells in the tumor that encode the first or second particular mutant epitope.

8. The method of claim 1 further comprising the step of testing one or more first samples and determining fraction of cells in the tumor that express the first or second particular mutant epitope.

9. The method of claim 1 wherein the somatic mutations comprise a single base substitution resulting in a single amino acid substitution.

10. The method of claim 1 wherein the somatic mutations comprise a frame shift mutation.

11. The method of claim 1 wherein the somatic mutations comprise a mutation resulting in an insertion or deletion of from 1-5 amino acid residues.

12. The method of claim 1 wherein the first sample is a liquid biopsy.

13. The method of claim 1 wherein the third sample is a liquid biopsy.

14. The method of claim 1 further comprising identifying the first particular mutant pllope as subject to loss of heterozygosity in the third sample.

15. The method of claim 1 further comprising identifying the first particular mutant epitope as subject to subclonal elimination from the tumor sample.

16. The method of claim 1 wherein the third sample is collected from the individual after the tumor begins to demonstrate resistance to the anti-tumor agent.

17. The method of claim 1 wherein the third sample is collected from the individual before the tumor begins to demonstrate resistance to the anti-tumor agent.

18. The method of claim 16 wherein the anti-tumor agent is a checkpoint inhibitor.

19. The method of claim 1 wherein the anti-tumor agent is a checkpoint inhibitor.

20. The method of claim 1 wherein the massively parallelsequencing is performed on the whole exome.

21. The method of claim 1 further comprising the step of making a peptide that comprises the second particular mutant epitope.

22. The method of claim 1 further comprising the step of delivering a peptide to the individual that comprises the second particular mutant epitope.

23. The method of claim 1 further comprising the step of making a peptide comprising the first particular mutant epitope.

24. The method of claim 1 further comprising the step of delivering to the individual a peptide that comprises the first particular mutant epitope and the second particular mutant epitope.

25. The method of claim 1 further comprising the step of delivering to the individual a peptide that comprises the first particular mutant epitope and a peptide that comprises the second particular mutant epitope.

26. The method of claim 22 further comprising the step of delivering an immune adjuvant o the individual.

27. The method of claim 24 further comprising the step of delivering an immune adjuvant to the individual.

28. The method of claim 1 further comprising the step of:
stimulating T cells of the individual in vitro with a peptide that comprises the second particular mutant epitope.

29. The method of claim 28 further comprising the step of:
expanding the T cells in vitro.

30. The method of claim 29 further comprising the step of:
re-infusing the T cells to the individual.

31. The method of claim 28 wherein the T cells are obtained from peripheral blood lymphocytes of the individual.

32. The method of claim I further comprising the step of:
making chimeric antigen receptor T cells that specifically bind to the second particular mutant epitope.

33. The method of claim 1 further comprising the step of :
delivering to the individual chimeric antigen receptor T cells that specifically bind to the second particular mutant epitope.

34. A personalized, anti-tumor immunogenic preparation customized for an individual cancer patient who initially responded to anti-tumor therapy and later became resistant to the therapy, comprising: a peptide that comprises a mutant epitope, and an adjuvant, wherein the mutant epitope is expressed in a tumor in the individual cancer patient, wherein the mutant epitope is recognized by a class I MHC molecule expressed by the individual cancer patient, and wherein the mutant epitope is present in the tumor after the tumor became resistant to the therapy.

35. The personalized, anti-tumor immunogenic preparation of claim 34 wherein the peptide is identified by the steps of:
performing massively parallel sequencing on a first sample of the individual comprising tumor DNA, on a second sample from the individual comprising normal tissue DNA, and on a third sample from the individual comprising tumor DNA, wherein the first sample is obtained prior to treatment with an anti-tumor agent and the third sample is obtained after treatment with the anti-tumor agent;
identifying somatic mutations in the first sample that encode a different amino acid sequence than in the second sample and form mutant epitopes;
analyzing the mutant epitopes in the first sample to identify epitopes that are recognized by class I MHC molecules of a type expressed by the individual; and
identifying from among the epitopes that are recognized by class I MHC molecules of a type expressed by the individual a first particular mutant epitope that is absent in the third sample and a second particular mutant epitope that is present in the third sample.

36. A personalized, anti-tumor, chimeric antigen receptor (CAR) customized for an individual cancer patient who initially responded to anti-tumor therapy and later became resistant to the therapy, comprising: a single chain variable region fragment that specifically binds to a mutant epitope, wherein the mutant epitope is expressed in a tumor in the individual cancer patient, wherein the mutant epitope is recognized by a class I MHC molecule expressed by the individual cancer patient, and wherein the mutant epitope is present in the tumor after the tumor became resistant to the therapy.

37. The personalized, anti-tumor, chimeric antigen receptor of claim 36 further comprising one co-stimulation domain.

38. The personalized, anti-tumor, chimeric antigen receptor of claim 36 further comprisingat least two co-stimulation domains.

39. The personalized, anti-tumor, chimeric antigen receptor of claim 36 further comprising at least three co-stimulation domains.

40. The personalized, anti-tumor, chimeric antigen receptor of claim 36 further comprising a signal peptide.

41. The personalized, anti-tumor, chimeric antigen receptor of claim 36 furthercomprising a transmembrane domain.

42. The personalized, anti-tumor, chimeric antigen receptor of claim 36 wherein the mutant epitope is identified by the steps of:
performing massively parallel sequencing on a first sample of the individual comprising tumor DNA, on a second sample from the individual comprising normal tissue DNA, and on a third sample from the individual comprising tumor DNA, wherein the first sample is obtained prior to treatment with an anti-tumor agent and the third sample is obtained after treatment with the anti-tumor agent;
identifying somatic mutations in the first sample that encode a different amino acid sequence than in the second sample and form mutant epitopes;
analyzing the mutant epitopes in the first sample to identify epitopes that are recognized by class I MHC molecules of a type expressed by the individual; and
identifying from among the epitopes that are recognized by class I MHC molecules of a type expressed by the individual a first particular mutant epitope that is absent in the third sample and a second particular mutant epitope that is present in the third sample.

43. A personalized, anti-tumor chimeric antigen receptor T cell customized for an individual cancer patient who initially responded to anti-tumor therapy and later became resistant to the therapy, wherein the personalized, anti-tumor, chimeric antigen receptor T cell comprises a chimeric antigen receptor (CAR) and the CAR comprises: a single chain variable region fragment that specifically binds to a mutant epitope, wherein the mutant epitope is expressed in a tumor in the individual cancer patient, wherein the mutant epitope is recognized by a class I MHC molecule expressed by the individual cancer patient, and wherein the mutant epitope is present in the tumor after the tumor became resistant to the therapy.

44. The personalized, anti-tumor, chimeric antigen receptor T cell of claim 43 wherein the chimeric antigen receptor comprises: one co-stimulation domain.

45. The personalized, anti-tumor, chimeric antigen receptor T cell of claim 43 wherein the chimeric antigen receptor comprises: at least two co-stimulation domains.

46. The personalized, anti-tumor, chimeric antigen receptor T cell of claim 43 wherein the chimeric antigen receptor comprises: at least three co-stimulation domains.

47. The personalized, anti-tumor, chimeric antigen receptor T cell of claim 43 wherein the chimeric antigen receptor comprises: a signal peptide.

48. The personalized, anti-tumor, chimeric antigen receptor T cell of claim 43 wherein the chimeric antigen receptor comprises: a transmembrane domain.

49. The personalized, anti-tumor, chimeric antigen receptor T cell of claim 43 wherein the mutant epitope is identified by the steps of:
performing massively parallel sequencing on a first sample of the individual comprising tumor DNA, on a second sample from the individual comprising normal tissue DNA, and on a third sample from the individual comprising tumor DNA, wherein the first sample is obtained prior to treatment with an anti-tumor agent and the third sample is obtained after treatment with the anti-tumor agent;
identifying somatic mutations in the first sample that encode a differentamino acid sequence than in the second sample and form mutant epitopes;
analyzing the mutant epitopes in the first sample to identify epitopes that are recognized by class I MHC molecules of a type expressed by the individual; and
identifying from among the epitopes that are recognized by class I MHC molecules of a type expressed by the individual a first particular mutant epitope that is absent in the third sample and a second particular mutant epitope that is present in the third sample.

50. A method of identifying target epitopes for a tumor of an individual, comprising:
performing massively parallel sequencing on a first liquid biopsy sample of the individual comprising tumor DNA and on a second liquid biopsy sample from the individual comprising tumor DNA, wherein the first sample is obtained prior to treatment with an anti-tumor agent and the second sample is obtained after treatment with the anti-tumor agent;
identifying somatic mutations in the first sample that encode a different amino acid sequence than encoded by normal DNA of the individual and that form mutant epitopes;
analyzing the mutant epitopes in the first sample to identify epitopes that are cognized by class I MHC molecules of a type expressed by the individual; and
identifying from among the epitopes that are recognized by class I MHC molecules of the type expressed by the individual a first particular mutant epitope that is absent in the second sample and a second particular mutant epitope that is present in the second sample.

51. The method of claim 50 further comprising the step of delivering a peptide to the individual that comprises the second particular mutant epitope.

52. The method of claim 50 further comprising the step of making a peptide comprising the first particular mutant epitope.

53. The method of claim 50 further comprising the step of delivering to the individual a peptide that comprises the first particular mutant epitope and the second particular mutant epitope.

54. The method of claim 50 further comprising the step of delivering to the individual a peptide that comprises the first particular mutant epitope and a peptide that comprises the second particular mutant epitope.

55. A method of treating a tumor in an individual comprising:
administering to the individual the personalized, anti-tumor immunogenic preparation of claim 34.

56. A method of treating a tumor in an individual comprising:
administering to the individual the personalized, anti-tumor chimeric antigen receptor of claim 36.

57. A method of treating a tumor in an individual comprising:
administering to the individual the personalized, anti-tumor chimeric antigen receptor T cell of claim 43.
